(19) United States
(12) Patent Application Publication
(10) Pub. No.: US 2004/0116870 A1
(43) Pub. Date: Jun. 17, 2004

(54) Marking syringe with liquid medicine bottle mount

(76) Inventor: Hogan Thomas, Powder Springs, GA (US)

Correspondence Address: TROUTMAN SANDERS LLP, BANK OF AMERICA PLAZA, SUITE 5200, 600 PEACHTREE STREET , NE, ATLANTA, GA, 30308-2216, US

(21) Appl. No.: 10/320,925

(22) Filed: Dec. 16, 2002

(51) Int. Cl. A61M 5/00

(52) U.S. Cl. 604/182

(57) ABSTRACT

An animal syringe assembly having a direct bottle mount for receiving and retaining a liquid medicine bottle. In one embodiment, the syringe assembly includes a medicine syringe having a plunger extending from an end of an elongated shaft into a medicine dosage chamber. The shaft includes an interior axial conduit extending substantially therethrough which communicates with the medicine dosage chamber. A handle for actuating the medicine syringe includes a direct mount for receiving and retaining a liquid medicine bottle. The bottle mount provides direct fluid communication from the liquid medicine bottle to the interior axial conduit of the shaft of the medicine syringe. Vaccine is drawn into the medicine dosage chamber from the liquid medicine bottle through the interior axial conduit.

### TECHNICAL FIELD

The present invention relates to syringes for vaccinating and marking animals. More particularly, the present invention relates to a marking syringe adapted to receive and retain a bottle of vaccine directly on the marking syringe.

### BACKGROUND INFORMATION

The regular and accurate administration of medicine to animals such as hogs and cattle is critical to the physical health of the animals. Complex medicinal regimens have been developed and implemented in an effort to generate healthier animals that produce a safe, high quality and quantity of product. Also, because meat protein is valued based on its quality characteristics, producers raising food animals for profit remain in search of the optimum medical regimen and the most cost effective application methods.

Numerous advances in medicine delivery syringes have helped producers administer medicines to food animals. For example, my U.S. Pat. No. 5,961,494, which is incorporated herein by reference, discloses a marking syringe which, when actuated, simultaneously injects medicine into an animal and places a color coordinated mark on the skin of the animal in the proximity of the injection. This marking syringe (known commercially as the “VAC-MARC” and/or “PRIMA-MARC”) reduces a two-step injecting and marking process into one step process of actuating the syringe.

Another advance includes a system for automatically recording animal injection information as disclosed in my U.S. Pat. No. 6,401,071, which is also incorporated herein by reference. A producer using a modified version of the syringe taught by my '494 may automatically record information relating to the administration of medicines to animals. While injecting and marking the animal, the syringe transmits a signal relating to the actuation of the syringe and the resulting injection of the animal. An electronic identification device on the animal provides the identification of the animal which is transmitted to a receiver. The receiver also receives the signal relating to the actuation of the syringe and the resulting injection of the animal. The animal is automatically identified upon delivery of the injection to the animal and then both the identity of the animal and the occurrence of the injection are recorded in a computer database. This information is then maintained in the computer database for review and analysis.

As shown in FIG. 1, known marking syringes may also include a mount 10 for receiving and retaining a liquid medicine bottle (not shown) directly to the medicine dosage chamber 12 of the medicine syringe 14 of the syringe assembly 18. Because the liquid medicine bottle held in the bottle mount 10 of the syringe assembly 18 is in direct fluid communication with the medicine dosage chamber 12, the vaccine from the bottle is permitted to pass directly into the medicine syringe 14.

A marking syringe having the liquid medicine bottle mounted directly to the medicine dosage chamber such as the syringe assembly 18 of FIG. 1, more easily provides a sterile mount to the medicine syringe. Also, the vaccine is not exposed to temperature fluctuations and UV light while in elongated tubing connected to the syringe and extending to a separately maintained liquid medicine bottle. Moreover, a direct connection decreases the chance of air getting into the marking syringe and then being injected into the animal causing significant discomfort and possible tissue damage.

The direct mounting of the liquid medicine bottle to a marking syringe also allows producers to have a direct and constant view of the supply of vaccine in the liquid medicine bottle. In other words, the producers can inventory the amount of vaccine in a simple glance.

However, when utilizing a bottle mount coupled directly to the medicine dosage chamber as best shown in FIG. 1, it is impossible to include a marking dispenser above the medicine syringe 14, such as disclosed in my U.S. Pat. No. 6,264,637, which is incorporated herein by reference. Another problem with the syringe assembly 18 is that mounting the liquid medicine bottle to the medicine dosage chamber 12 puts a significant amount of weight on the front of the syringe assembly 18. Also, because the liquid medicine bottle is forward of the handle of the syringe assembly 18, the liquid medicine bottle is vulnerable to damage from impacts. As a result, the syringe assembly 18 is not ergonomically balanced and is prone to impact damage. The connection between the liquid medicine bottle and the dosage chamber is weak and will leak vaccine when damaged.

Therefore, there is a need for an improved marking syringe having an auto-reloading vaccine chamber, marking dispenser, and a direct mount to the marking syringe for receiving and retaining the liquid medicine bottle. However, the direct mount must permit the liquid medicine bottle and the marking dispenser to balance against one another to provide an ergonomically balanced marking syringe. Also, the improved marking syringe must be adapted to more thoroughly protect the liquid medicine bottle from impact and exposure to detrimental outside factors such as temperature and ultraviolet light exposure.

### BRIEF SUMMARY OF THE INVENTION

The present invention solves the above-identified problems by providing a syringe assembly having an improved direct mount for the sterile receipt of medicine and for the sterile receipt of medicine and for retaining the medicine bottle. The direct bottle mount extends from the rear of the syringe assembly rather than the front.

Generally described, the present invention includes a syringe assembly having a direct bottle mount for receiving and retaining a liquid medicine bottle. The syringe assembly includes an auto-reloading medicine syringe having a plunger extending from an end of an elongated shaft into a medicine dosage chamber. The shaft includes an interior axial conduit extending substantially therethrough which communicates with the medicine dosage chamber. A handle for actuating the medicine syringe supports a direct mount for receiving and retaining a liquid medicine bottle. The bottle mount provides direct fluid communication from the liquid medicine bottle to the interior axial conduit of the hollow shaft of the vaccine syringe. Vaccine is drawn into the medicine dosage chamber from the medicine bottle through the interior axial conduit.

According to one aspect of the invention, the syringe assembly further includes a dispenser such as an ink dispenser for marking the animal in the proximity of the injection site.

The foregoing has broadly outlined some of the more pertinent aspects and features of the present invention. These should be construed to be merely illustrative of some of the more prominent features and applications of the invention. Other beneficial results can be obtained by applying the disclosed information in a different manner or by modifying the disclosed embodiments. Accordingly, other aspects and a more comprehensive understanding of the invention may be obtained by referring to the detailed description of the exemplary embodiments taken in conjunction with the accompanying drawings, in addition to the scope of the invention defined by the claims.

### BRIEF DESCRIPTION OF THE DRAWINGS

FIG. 1 depicts a prior art embodiment of a syringe assembly having a bottle mount coupled directly to the medicine dosage chamber of a auto reloading syringe.

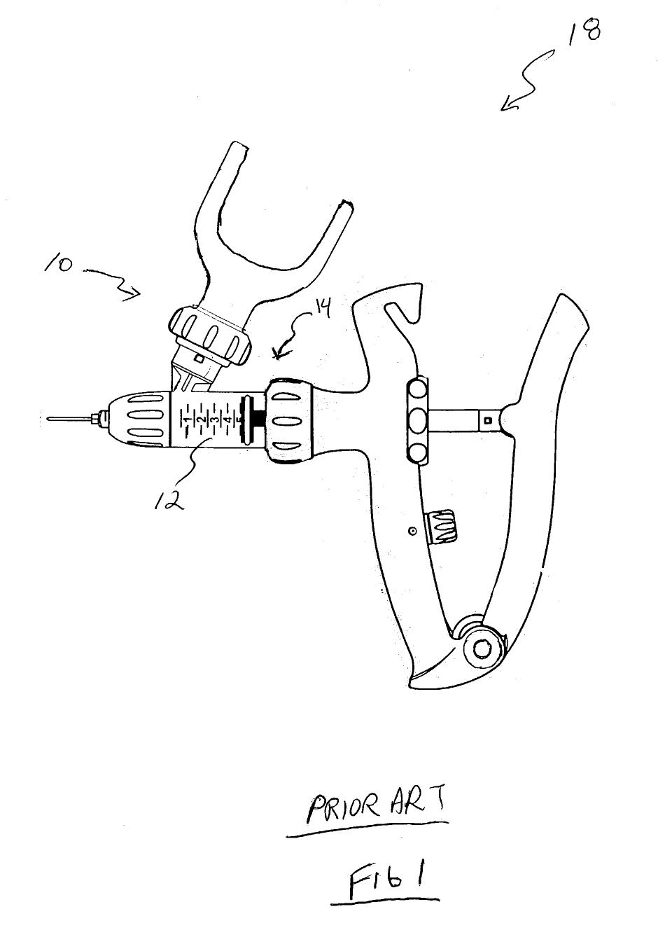

FIG. 2 depicts an exemplary embodiment of the syringe assembly of the present invention having a marking device and an improved bottle mount.

FIG. 3 depicts an exploded view of one embodiment of a portion of the handle, bottle mount and plunger of the present invention.

FIG. 4 depicts the syringe assembly of FIG. 2 without a pressurized canister being held within a carriage and having a portion of the handle cut away.

FIG. 5 depicts a partial top view of one embodiment of the bottle mount and shaft of the present invention taken along line A-A of FIG. 4.

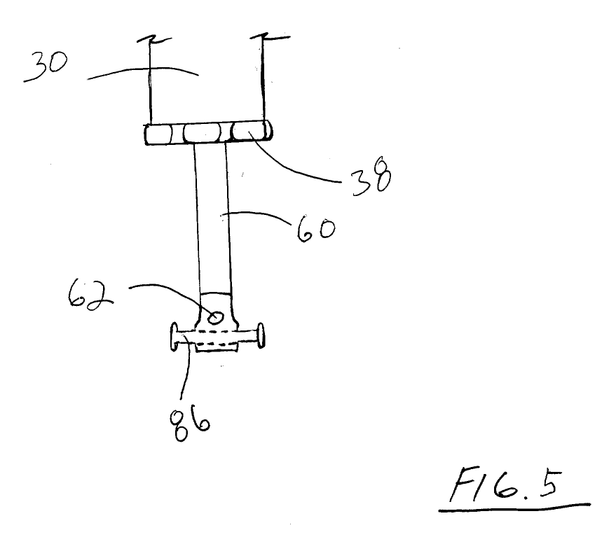

### DETAILED DESCRIPTION

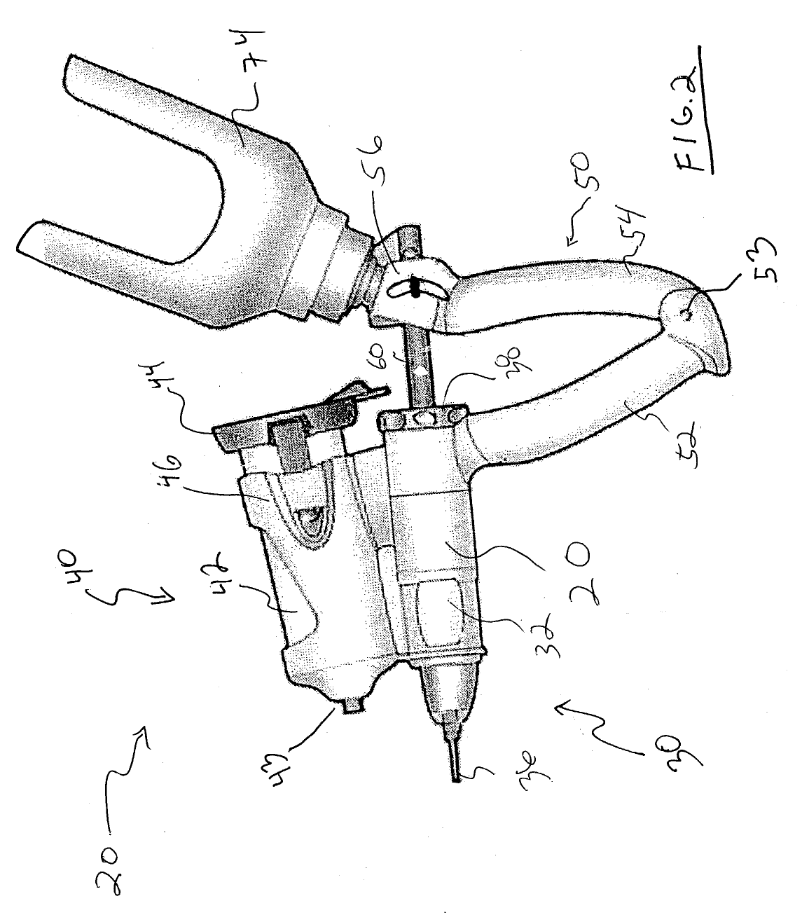

FIG. 2 depicts an exemplary embodiment of a syringe assembly 20 of the present invention. The syringe assembly 20 may be a transceiving syringe having the ability to simultaneously deliver an injection to the animal and deliver a marking spot to the animal. The syringe assembly may also have the ability to receive identification information from an electronic identification device (EID) such as a read/write radio frequency identification device (RWRFID) attached to the animal, relay identification information relating to the animal and injection information relating to the delivery of the injection to a database as “event information”, and transmit the event information to the RWRFID for redundant storage thereon. Many of the essential functions of a transceiving syringe are known to those skilled in the prior art due to my U.S. Pat. Nos. 5,961,494 and 6,264,637 directed to marking syringes and U.S. Pat. No. 6,401,071 directed to a system and method for automatically recording animal injection information.

In a preferred embodiment of the present invention, the syringe assembly 20 includes a medicine syringe 30 and a dispenser 40 for applying an indicator such as ink in close proximity to the location of the injection to the animal. The dispenser 40 is preferably includes a pressurized canister 42 held by a can détente 44 and carriage 46, which cooperate with one another to retain the canister 42, as disclosed in my U.S. patent application having Ser. No. 09/848,495, entitled “Can Détente”, filed May 3, 2001, incorporated herein by reference. However, the dispenser 40 may take any number of forms well known to those skilled in the art.

The medicine syringe 30 preferably includes a medicine dosage chamber 32, a plunger 34 and an outwardly extending needle 36. Alternatively, needle-less injections may be used. The medicine dosage chamber 32 is adapted for receiving the plunger 34. The amount of vaccine which may be received into the medicine dosage chamber 32 for a single injection preferably is varied by rotating the dose adjuster 38 as disclosed in my U.S. patent application having Ser. No. 09/872,547, entitled “Orientationally Adjustable Marking Syringe”, filed Jun. 2, 2001, incorporated herein by reference. Also, the syringe assembly 20 preferably has the capabilities of permitting the dispenser 40 to pivot about the medicine syringe 30 as disclosed in my '547 patent application.

The syringe assembly 20 also includes a handle 50 for actuating the medicine syringe and marking dispenser. Preferably, the handle 50 includes a first handle portion 52 and a second handle portion 54 which are pivotally connected to one another at pivot point 53. As shown in FIG. 2, the first handle portion 52 captures the medicine syringe 30 and the dispenser 40. The second handle portion 54, on the other hand, actuates the medicine syringe 30 and the dispenser 40 when the handle 50 is actuated.

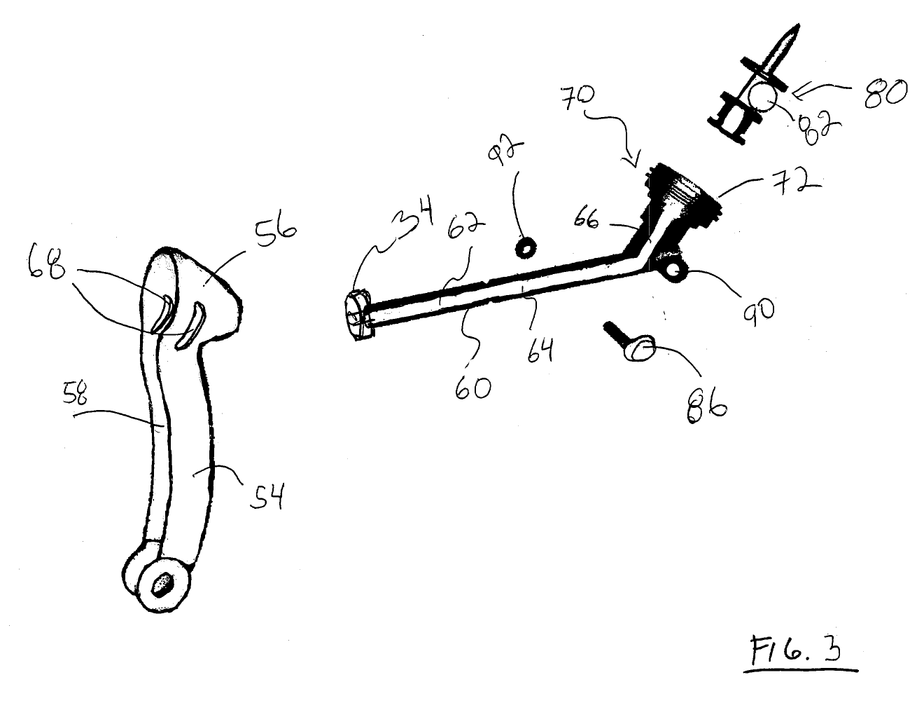

Also, as best illustrated in FIG. 3, the plunger 34 extends from an end of an elongated shaft 60 which includes an interior axial conduit 62 extending substantially therethrough. The interior axial conduit 62 communicates with the dosage chamber 32 when the syringe assembly 20 is assembled for use. Preferably, the interior axial conduit 62 is rigid and includes a first portion 64 and a second portion 66 which are angled relative to each other. As shown in FIG. 3, the second portion 66 is oriented upward relative the first portion 64.

The syringe assembly 20 further includes a sterile direct mount 70 for receiving and retaining a liquid medicine bottle (not shown) or other container suitable for holding and dispensing vaccine. The direct mount 70 is supported by a top end 56 of the second handle portion 54 and permits direct fluid communication from the liquid medicine bottle to the interior axial conduit 62 of the shaft 60. Because the direct mount 70 is positioned behind the medicine syringe 30, the syringe assembly 20 is ergonomically balanced during use. The direct mount 70 saves time and labor because it improves efficiency of the cattleman.

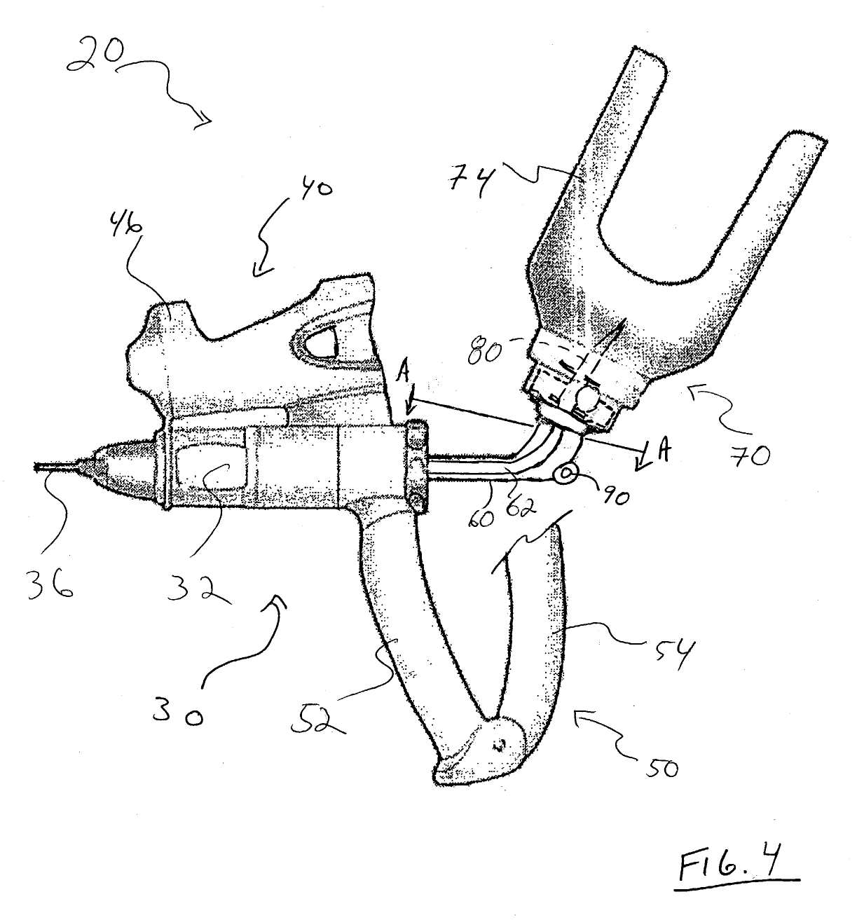

Preferably, the direct bottle mount 70 is configured to receive and retain the liquid medicine bottle by inserting a rubber stopper (not shown) of the liquid medicine bottle into an enlarged open end 72 of the direct mount 70 which is sized to receive the circumference of the rubber stopper. The exterior of the end 72 is threaded to receive a carrier 74 as shown in FIGS. 2 and 4. The carrier 74 may also be what is called a fast-fit bottle mount having a pair of semi-rigid and elongated retention grips which helps retain the liquid medicine bottle on the end 72 of the direct mount 70. In order to remove the liquid medicine bottle, the retention clips are squeezed together at their upper ends so that their lower ends pivot away from contact with the bottle in order to allow the liquid medicine bottle to be pulled from the syringe assembly 20. Alternatively, the carrier 74 may be snap fit on to the end 70.

As shown in FIGS. 2 and 3, the direct mount 70 would orient the liquid medicine bottle in a substantially inverted manner providing improved medicine flow into and through the direct bottle mount 70, the shaft 60, and the dosage chamber 32. Because of the direct mount 70, the syringe assembly 20 is free of flexible hose or conduit connected to a medicine reservoir, such as those retained on an arm, leg, or in a backpack-type retention device, typically used to provide vaccine to syringe assemblies.

When the syringe assembly 20 of the present invention is actuated by squeezing the handle 50, vaccine is drawn into the medicine dosage chamber 32 from the liquid medicine bottle through the interior axial conduit 62. Also, when the syringe assembly is actuated, the direct mount 70 moves forward toward the needle 36 in a corresponding manner with the shaft 60 and plunger 32 of the medicine syringe 30. In other words, the direct mount 70 preferably moves in substantially a corresponding manner with the second handle portion 54 when the handle 50 is squeezed to actuate the syringe assembly 20.

The first handle portion 52 also includes a concave side portion 58 as best shown in FIG. 3. The concave side portion 58 is adapted to receive at least a portion of the shaft 60. A pair of openings 68 extend through the handle portion 52 from the concave side portion 58 to another side of the first handle portion 52 such as the exterior of the first handle portion 52. The openings 68 are configured to receive a pin 86 which passes through an opening 90 in the shaft 60. The opening 90 is substantially transverse to the interior axial conduit 62, but does not communicate with the interior axial conduit 62. The pin 86 is held is place by fastener 92.

The shape of the openings 68 permit the pin 86 coupled to the shaft 60 to pivot within the openings 68 as the handle 50 is squeezed to actuate the syringe assembly 20. Preferably the openings 68 are slot-shaped. The shaft 60 is therefore permitted to move forward by the second handle portion 54 without being flexed and to insure axial alignment shaft 60 having plunger 34 with the medicine dosage chamber 32. The liquid medicine bottle in the direct mount 70 is also maintained in a consistent position as it is moving forward during the actuation of the syringe assembly 20.

As best shown in FIGS. 3 and 4, the direct mount 70 includes a breather draw spike 80 adapted for being received and retained in the direct mount 70. The draw spike 80 is preferably snap-fitted with O-ring seals into the end 72 of the direct mount 70. However, the spike may also be screwed into the end 72. The draw spike 80 is also adapted to extend into the carrier 74 and be received through the pierced rubber stopper on the liquid medicine bottle. The spike 80 facilitates the passage of vaccine from the liquid medicine bottle into the interior axial conduit 62 of the shaft 60. The spike 80 may include an air inlet breather valve 82 as shown in FIG. 3 as well as an internal removable filter. The filter may be used to remove debris, sediment and foreign objects from low quality or poorly hydrated medicine.

The present invention has been illustrated in relation to particular embodiments which are intended in all respects to be illustrative rather than restrictive. Those skilled in the art will recognize that the present invention is capable of many modifications and variations without departing from the scope of the invention. Accordingly, the scope of the present invention is described by the claims appended hereto and supported by the foregoing.

I claim:

1. A syringe assembly for vaccinating an animal comprising:
a medicine syringe having a plunger extending from an end of an elongated shaft into a medicine dosage chamber, said medicine dosage chamber adapted for receiving said plunger, and said shaft having an interior axial conduit extending substantially therethrough which communicates with said medicine dosage chamber;
a handle for capturing said medicine syringe, said handle allowing actuation of said medicine syringe; and
a direct mount for receiving and retaining a liquid medicine bottle, said direct mount extending from said handle and permitting direct fluid communication from the liquid medicine bottle to said interior axial conduit of said shaft of said medicine syringe, wherein vaccine is drawn into said medicine dosage chamber from the liquid medicine bottle through said interior axial conduit.

2. The syringe assembly of claim 1 wherein said direct mount is configured to orient the liquid medicine bottle in a substantially inverted manner when received and retained therein.

3. The syringe assembly of claim 1 wherein said direct mount moves in a corresponding manner with said shaft and said plunger of said medicine syringe when said syringe assembly is actuated to inject medicine into the animal.

4. The syringe assembly of claim 3 wherein said direct mount moves forward toward a needle extending from said medicine syringe when said syringe assembly is actuated to vaccinate the animal.

5. The syringe assembly of claim 1 being free of flexible hose means coupled to said medicine syringe for providing vaccine to said syringe assembly.

6. The syringe assembly of claim 1 wherein said handle comprises a first handle portion for capturing said medicine syringe and a second handle portion for actuating said medicine syringe.

7. The syringe assembly of claim 6 wherein said direct mount moves in substantially a corresponding manner with said second handle portion when actuating said medicine syringe.

8. The syringe assembly of claim 6 wherein said first handle portion and said second handle portion are pivotally connected to one another.

9. The syringe assembly of claim 1 wherein said direct mount includes a spike adapted for being received and retained therein and for permitting vaccine to pass from the liquid medicine bottle into said interior axial conduit of said shaft of said medicine syringe.

10. The syringe assembly of claim 1 wherein said handle is positioned substantially directly beneath the liquid medicine bottle received and retained in said direct mount.

11. The syringe assembly of claim 1 wherein said handle comprises a first handle portion for capturing said medicine syringe and a second handle portion for actuating said medicine syringe, said second handle portion having a concave side portion adapted to receive at least a portion of said shaft and at least one opening in said second handle portion extending through said second handle portion from said concave side portion to another side of said second handle portion, said opening configured to receive a pin coupled to said shaft and adapted to permit said pin to pivot therein when said shaft is moved forward when said syringe assembly is actuated.

12. The syringe assembly of claim 1 wherein said interior axial conduit extending through said shaft of said medicine syringe comprises a first portion and a second portion, wherein said second portion is angled from said first portion.

13. The syringe assembly of claim 12 wherein said second portion of said interior axial conduit is oriented upward relative said first portion of said interior axial conduit.

14. The syringe assembly of claim 1 wherein said shaft having said interior axial conduit therein is substantially rigid.

15. The syringe assembly of claim 1 wherein said direct mount and said handle cooperate to permit the liquid medicine bottle to maintain a constant orientation relative said syringe assembly when said syringe assembly is actuated.

16. The syringe assembly of claim 15 wherein the liquid medicine bottle is maintained in a substantially inverted manner.

17. The syringe assembly of claim 1 further comprising a dispenser for applying an indicator to the animal, said handle allowing actuation of both the said medicine syringe and said dispenser.

18. A marking syringe assembly for vaccinating an animal and applying an indicator in the general vicinity of the injection site, said marking syringe assembly comprising:
a dispenser for applying an indicator to the animal;
a medicine syringe having a plunger extending from an end of an elongated shaft into a medicine dosage chamber, said medicine dosage chamber adapted for receiving said plunger, and said shaft having an interior axial conduit extending substantially therethrough which communicates with said medicine dosage chamber;
a handle for capturing said medicine syringe and said dispenser, said handle allowing actuation of both said medicine syringe and said dispenser; and
a direct mount for receiving and retaining a liquid medicine bottle, said direct mount extending from said handle and permitting direct fluid communication from the liquid medicine bottle to said interior axial conduit of said shaft of said medicine syringe, said marking syringe assembly being free of flexible hose means coupled to said medicine syringe for providing vaccine to said marking syringe assembly, said direct mount moving in a corresponding manner with said shaft and said plunger of said medicine syringe when said marking syringe assembly is actuated to vaccinate and mark the animal, wherein medicine is drawn into said medicine dosage chamber from the medicine bottle through said interior axial conduit.

19. The marking syringe assembly of claim 18 wherein said handle comprises a first handle portion for capturing said medicine syringe and said dispenser and a second handle portion for actuating said medicine syringe and said dispenser.

20. A direct mount coupling a medicine bottle and a syringe assembly together, said direct mount extending from a handle of said syringe assembly and permitting fluid communication from the medicine bottle to a dosage chamber of said syringe assembly, said dosage chamber automatically reloading with medicine from said medicine bottle due to actuation of said syringe assembly.
